(19) United States
(12) Patent Application Publication
(10) Pub. No.: US 2007/0239126 A1
(43) Pub. Date: Oct. 11, 2007

(54) Absorbent article printed using digital printing technology and a method of printing

(75) Inventors: Christopher Michael Wilson, Appleton, WI (US); Andrew Peter Bakken, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/401,827

(22) Filed: Apr. 11, 2006

(51) Int. Cl. B41M 5/00; A61F 13/15

(52) U.S. Cl. 604/385.01; 428/195.1; 428/32.16

(57) ABSTRACT

A strip of absorbent material capable of being divided into a plurality of absorbent articles is disclosed. Each of the absorbent articles has a first major surface and at least one distinct image printed on the first major surface using digital printing technology. The distinct image printed on one of the absorbent articles is positioned at a different location relative to the distinct image printed on an adjacent absorbent article. A method of printing at least distinct image on a first major surface of a strip of absorbent material using digital printing technology is also disclosed.

### BACKGROUND OF THE INVENTION

Today, processes used to print images and text on folded and rolled absorbent articles, such as facial tissue, bath tissue, paper towel, wipes, etc., are very limited. The use of printing plates and plate rolls restricts the number of images that can be printed on the finished article, as well as the distance between repeating images. The fixed length of a printing plate and the fixed diameter of a plate roll create a predetermined image repeat length. In addition, a printing plate does not allow the printing system to offer a multitude of distinct images. In order to change images, one must replace the printing plate or plate roll with another plate or roll, respectively, having different images. This change requires shutting down the equipment, substituting one plate or roll for another and making a number of mechanical adjustments. Furthermore, the current printing processes do not allow the distinct images to be varied in size, location and registration as the pattern is repeated.

Digital printing is a relatively new technology that may be utilized in printing variable images on folded and rolled absorbent articles. Digital printing provides flexibility in image size and selection and also allows adjustability of the image registration on the finished article in both the machine and cross directions. Digital printing may also provide the opportunity to offer next generation printed absorbent articles in a cost effective and efficient manner.

Currently, manufacturers of facial tissue, bath tissue, paper towels, wipes, etc. have a strong desire to be able to sell a package of folded or rolled absorbent articles, wherein each article or sheet has a different image printed on it. Manufacturers would also like to offer such printed absorbent articles in multiple color images in a cost effective and timely manner.

Now, absorbent articles can be printed with distinct images using digital technology such that the size, position, location and registration of each adjacent image can be varied. In addition, all of the images can be distinct and a repeating pattern can be established for every n absorbent article contained within a package. Alternatively, a non-repeating pattern of images can be utilized.

### SUMMARY OF THE INVENTION

Briefly, this invention relates to a strip of absorbent material capable of being divided into a plurality of absorbent articles. Each of the absorbent articles has a first major surface and at least one distinct image printed on the first major surface using digital printing technology. The distinct image printed on one of the absorbent articles being positioned at a different location relative to the distinct image printed on an adjacent absorbent article.

A method of printing at least one distinct image on a first major surface of a strip of absorbent material that is capable of being divided into a plurality of absorbent articles, using digital printing technology, is also disclosed.

### BRIEF DESCRIPTION OF THE DRAWINGS

FIG. 1 is a top view of a strip of absorbent material separated into individual sheets by spaced apart lines of perforations wherein each sheet contains a distinct image printed using digital technology and the images are registered to one of the lines of perforations.

FIG. 2 is a side view of an individual absorbent article.

FIG. 3 is a top view of an alternative embodiment showing a strip of absorbent material separated into individual absorbent articles by spaced apart lines of perforations wherein each absorbent article contains a distinct image printed using digital technology and the images cross one of the lines of perforations and a repeating pattern of different size images is presented.

FIG. 4 is a top view of a strip of absorbent material separated into individual absorbent articles by spaced apart lines of perforations wherein each absorbent article contains a distinct image printed using digital technology and the distinct image on one of the absorbent articles is not in registration with an adjacent image.

FIG. 5 is a top view of a strip of absorbent material separated into individual absorbent articles by spaced apart lines of perforations wherein each absorbent article contains a distinct image printed using digital technology and the images exhibit variable registration relative to an adjacent image.

FIG. 6 is a top view of a strip of absorbent material separated into individual absorbent articles by spaced apart lines of perforations wherein each absorbent article contains an image printed using digital technology and the images exhibit variable registration in the machine and cross directions.

FIG. 7 is a top view of a strip of absorbent material separated into individual absorbent articles by spaced apart lines of perforations wherein some of the distinct images printed using digital technology cross the lines of perforations.

FIG. 8 is a schematic showing a strip of absorbent material having distinct images printed thereon using digital technology wherein the strip of absorbent material is cut into individual members, folded and stacked into a package.

FIG. 9 is a flow diagram depicting the transfer of multiple images using digital technology onto an absorbent article.

### DETAILED DESCRIPTION

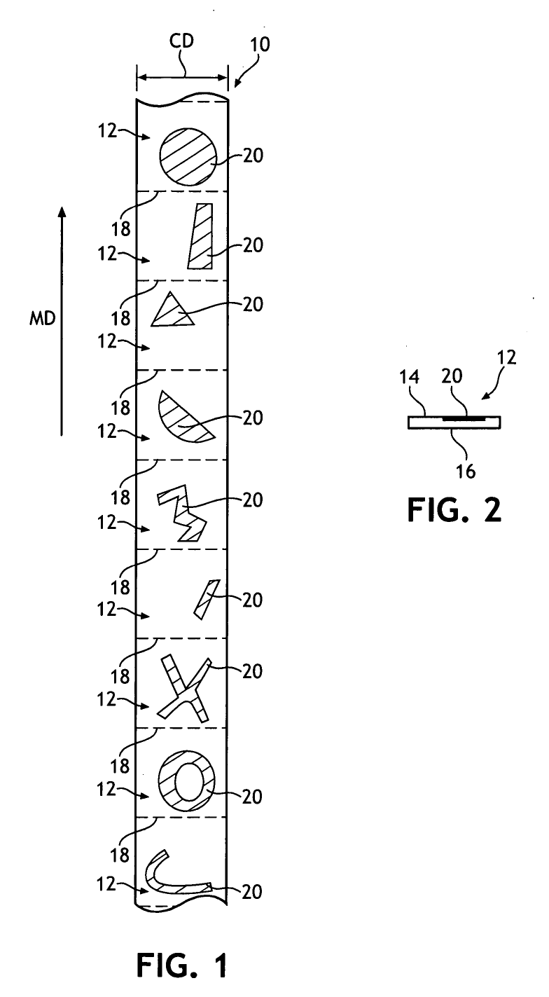

Referring to FIG. 1, a strip of absorbent material 10 is shown which is capable of being divided into a plurality of absorbent articles 12. By “absorbent” it is meant that the strip of material 10 is constructed from a material that is capable of absorbing a liquid or a fluid. An example of a liquid is water. By “capable of being divided” it is meant that the absorbent articles 12 can be manufactured in such a fashion that they are connected together but can be easily broken, torn apart or otherwise separated from one another at a later point in time. For example, a roll of bath tissue can contain a plurality of absorbent sheets each being connected by spaced apart lines of perforations which allow the sheets to be separated at any desired length when needed. By “plurality” it is meant three or more absorbent articles 12. Desirably, a plurality of absorbent articles will include at least 20 articles. More desirably, a plurality of absorbent articles will include at least 50 articles. Even more desirably, a plurality of absorbent articles will include at least 100 articles.

The strip of absorbent material 10 can be formed from natural and/or synthetic fibers. Desirably, the absorbent material 10 can be made from cellulosic fibers, textile fibers, from wood pulp, from a combination of fibers and pulp, or from any other absorbent materials known to those skilled in the art. Synthetic fibers, such as thermoplastic fibers formed from polypropylene or polyethylene can also be used. When synthetic fibers are utilized, they most likely will have to be chemically coated or treated to make them hydrophilic. By “hydrophilic” it is meant that the material has an affinity for absorbing a liquid or a fluid. It is also possible to mix cellulosic fibers with synthetic fibers to form a durable absorbent material. Furthermore, a superabsorbent can be added to the cellulosic fibers to increase the absorbent capacity of the absorbent articles. A superabsorbent is a material having a high capacity for absorbing and retaining a liquid or a fluid. Superabsorbents are commonly employed in solid form and can vary in shape. Typically, small particles, granules, flakes, etc. are utilized. If one wishes to incorporate a superabsorbent, it can be mixed, combined, attached, printed or otherwise added to the strip of absorbent material 10.

The strip of absorbent material 10, shown in FIG. 1, has a machine direction, denoted as MD, and a cross direction, denoted CD. The cross direction is aligned perpendicular to the machine direction.

Referring to FIG. 2, an absorbent article 12 is depicted as an individual sheet having a first major surface 14 and a second major surface 16. The first and second major surfaces, 14 and 16 respectively, can be aligned opposite to one another. By a “major surface” it is meant a surface having sufficient area relative to a narrow end or edge which can have an image printed thereon using digital technology. By “digital printing technology” it is meant a printer, such as an ink jet printer, that transfers images consisting of pixels to a controller electronically. The computer will have access to a database containing a variety of distinct images that can be selected and conveyed to the controller which in turn controls the printer such that the images can be printed on the absorbent articles at a predetermined location. Desirably, the printer does not contact the sheet so that the intrinsic sheet properties are not modified. Kodak Versamark, Inc., is one company that commercially sells digital technology equipment and processes for printing various substrate materials. Kodak Versamark, Inc., has an office at 3000 Research Blvd., Dayton, Ohio 45420. Additional information on digital technology will be explained later.

The absorbent article 12 can be almost any type of absorbent product that is manufactured as an individual article, such as a sheet, or as a plurality of articles temporarily connected together, such as a roll of toilet paper connected by lines of perforations. Examples of individual absorbent articles include but are not limited to facial tissue, paper napkins, dry wipes, wet wipes, industrial wipers, etc. Examples of absorbent articles formed as a roll include but are not limited to a roll of bath tissue, commonly referred to as a roll of toilet paper, a roll of paper towels, a roll of wipes, etc. Furthermore, the absorbent article 12 can have a flat appearance or it can be folded, creased, corrugated or configured to have a topographical profile or a non-planar configuration.

Returning again to FIG. 1, the strip of absorbent material 10 is divided into a plurality of continuous absorbent articles 12 connected together by breakable lines of perforations 18. Each of the lines of perforations 18 extend transversely across the strip of absorbent material 10, parallel to the cross direction CD. However, the lines of perforations 18 can be alternatively formed at an angle to the cross direction CD such that they are no longer aligned perpendicular to the machine direction MD. The lines of perforations 18 do not entirely separate one absorbent article 12 from another but instead provide an easy means for a consumer to tear or separate the absorbent articles 12 at a later point in time. It should be noted that the consumer does not have to separate each absorbent article 12 from an adjacent absorbent article 12 but, instead, can elect to keep several of the absorbent articles 12 attached together so that they are easier to use for their intended purpose. A good example of this is when a person pulls out a strip of toilet paper from the roll but does not separate the strip until six or seven sheets are advanced.

Still referring to FIG. 1, the absorbent articles 12 can be in the form of a continuous sheet, such as a roll of bath tissue, a roll of paper towels, a roll of wipes, etc. wherein each absorbent article 12 is temporarily connected together by one of the lines of perforations 18. The lines of perforations 18 are frangible and are designed to be easily broken, torn or separated. Once a line of perforations 18 is broken, an individual absorbent article 12 is obtained. Alternatively, instead of using lines of perforations 18, a manufacturer may select to cut, sever or divide the strip of absorbent material 10, at predetermined locations, into individual absorbent articles 12 before the absorbent articles are sold to the public. These individual absorbent articles 12 can then be folded, stacked and/or otherwise manipulated in some fashion to form a finished absorbent article 12. Two or more individual absorbent articles 12 can be packaged together for sale to the ultimate consumer. An example of individual absorbent articles 12 that have been cut, folded, stacked and packaged in a single container is a box of facial tissue.

Returning to FIG. 2, the absorbent article 12 can consist of a single layer or ply as shown. Alternatively, the absorbent article 12 can be constructed of two, three or more layers, if desired. Some bath tissue is manufactured with up to 5 layers. Normally, most bath tissue, paper towels, facial tissue and wipes consist of from 1 to 4 layers. Two and three layers are very common. Premium absorbent articles tend to use multiple layers to deliver enhanced softness and absorbency.

Referring now to FIGS. 1 and 2, each of the absorbent articles 12 has at least one image 20 printed thereon using digital technology. Optionally, every absorbent article 12 does not have to have any image 20 printed thereon. Some absorbent articles 12 can be without an image or may only have a portion of an image 20 printed thereon. For example, it is possible using digital technology to print on one absorbent article 12 and then skip the next four or five absorbent articles and print again on the sixth absorbent article. It is also possible using digital technology to print a portion of an image on one absorbent article 12 and the remainder of the image on an adjacent absorbent article 12. The printed image 20 can be formed using various kinds of non-impact printers and methods known to those skilled in the art. The most common non-impact printer useful for printing on an absorbent article 12 is an ink jet printer. An ink jet printer can utilize either water base ink, hot melt ink or solvent based ink. A water based ink works well in printing images on a strip of absorbent material 10. However, a solvent base ink could also be employed, if desired. The ink jet printer can be of any type currently sold including a piezoelectric printer, a valve jet printer, a continuous ink jet or a thermal printer.

The strip of absorbent material 10 can be printed before it is wound up into a cylindrical roll (not shown) or it can be printed after it has been unwound from a supply roll. The exact point in the process where the strip of absorbent material 10 is printed does not matter so long as the strip of material 10 is sufficiently dry and can soak up the ink that is applied to it and sufficient time is provided to allow the ink to dry.

The image 20 should be printed on at least one of the first or second major surfaces, 14 and 16 respectively, of the absorbent article 12. Each image 20 should be clearly visible on the first major surface 14. For some absorbent articles, it may be desirous to have the image 20 be clearly visible on both of the first and second major surfaces, 14 and 16 respectively. This can be accomplished by printing the image 20 on both of the first and second major surfaces, 14 and 16 respectively, or printing the image 20 on one surface and allowing the ink to penetrate through to the opposite surface.

In FIG. 1, each of the images 20 is a distinct image which varies from each adjacent image 20. In addition, each of the distinct images 20 is depicted as being visually different from all of the remaining images 20 formed on the strip of absorbent material 10. By “distinct” it is meant that the image 20 is distinguishable from all the other images 20 and is easily perceived by the senses or intellect. In FIG. 1, there is no repeating pattern of images 20 on the strip of absorbent material 10. In addition, each of the distinct images 20 is in a different position or location on each of the absorbent articles 12 relative to an adjacent distinct image 20. Furthermore, one will also notice that the distinct images 20 also vary in size and shape and the registration in the machine and cross directions also varies. If desired, any of these variables can remain constant.

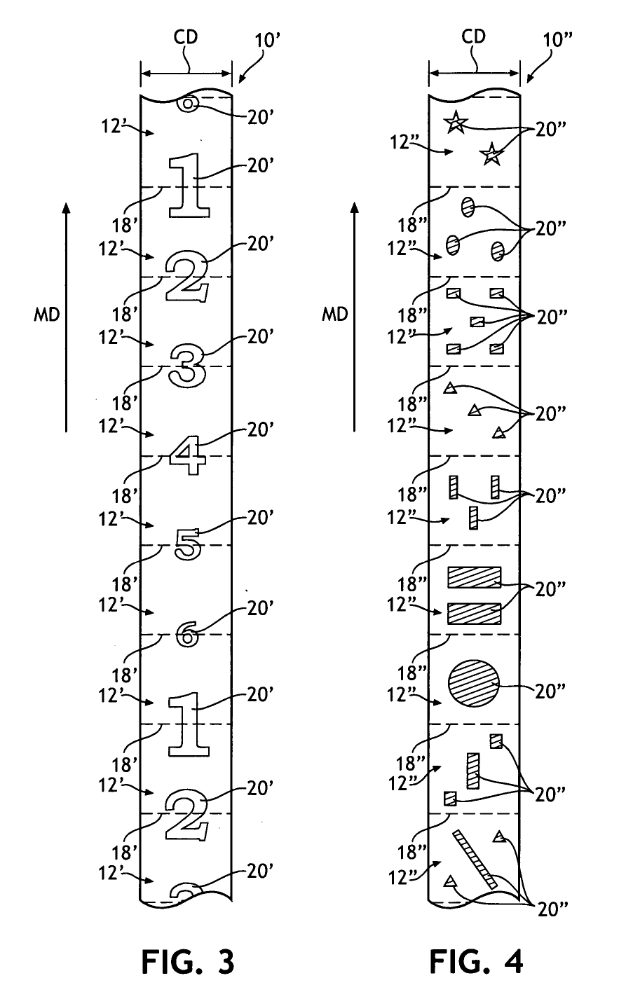

FIGS. 3-7 depict several alternative embodiments of images formed on a strip of absorbent material. The strips of absorbent materials can represent a roll of bath tissue, a roll of paper towels, a roll of wipes, etc. Referring now to FIG. 3, a strip of absorbent material 10′ is shown which is capable of being divided into a plurality of absorbent articles 12′ along lines of perforations 18′. Each of the absorbent articles 12′ contains an image 20′ which creates a repeating pattern of distinct images 20′ every n absorbent article, wherein n is greater than 6. It should be noted that n can be any integer greater than 2 to infinity. Desirably, n is greater than 10. More desirably, n is greater than 50. Still more desirably, n is greater than 80. Most desirably, n is greater than 100.

In FIG. 3, one will also notice that each of the first six images 20′ has a different size. In FIG. 3, number 1 is larger than number 2, etc., up to number 6. The pattern then repeats itself after number 6, starting again with a large size number 1. Furthermore, in FIG. 3, each of the images 20′ is printed such that a portion of the image 20′ extends across one of the lines of perforations 18′. This is different from FIG. 1, wherein each image 20 is registered so as to be spaced apart from the lines of perforations 18. By printing images 20′ using digital technology, one can vary the images 20′, the size of the various images 20′, the position or location of the various images 20′, as well as control the machine direction and cross direction registration of each of the images 20′.

Because of the size difference of each image 20′ depicted in FIG. 3, one will notice that each image 20′ is positioned at a different location on each of the n absorbent articles 12′ relative to an adjacent image 20′. It should be noted that it is also possible to print a distinct image 20′, using digital technology, on one of the absorbent articles 10′ such that it is in registration with a distinct image 20′ printed on an adjacent absorbent article 12′ but still is of a different size.

Referring now to FIG. 4, another embodiment is shown of a strip of absorbent material 10″ capable of being divided into a plurality of absorbent articles 12″ along lines of perforations 18″. Each of the absorbent articles 12″ contains one or more images 20″ printed using digital technology. The images 20″ do not create a repeating pattern along any portion of the length of the strip of absorbent material 10″. However, the pattern could be repeated when the number of absorbent articles exceed ten or more, if desired. In FIG. 4, one will readily notice that from one to five images 20″ are printed on each of the absorbent articles 12″. The size, shape, position or location, and machine and cross direction registration of each of these distinct images 20″ is different from the remaining images 20″. The ability to print variable and diverse images 20″ on each adjacent absorbent article 12″, as well as to vary the frequency of a repeatable pattern, is now possible using digital technology.

Turning now to FIG. 5, still another embodiment is shown of a strip of absorbent material 10′″ capable of being divided into a plurality of absorbent articles 12′″ along lines of perforations 18′″. Each of the absorbent articles 12′″ contains an image 20′″. The images 20′″ are shown as individual letters. The images 20′″ do not represent a repeating pattern of distinct images 20′″. However, since only seven complete absorbent articles 12′″ are shown, it is possible to form a repeating pattern at every eighth or greater absorbent articles 12′″, if desired. It should be noted that the number of absorbent articles 12′″ that are present is less than the number of absorbent articles 12′″ needed to represent a repeating pattern such that, desirably, no repeating pattern is visible to the ultimate consumer.

In FIG. 5, one will readily notice that a single image 20′″, in the form of a capital letter, is printed on each of the absorbent articles 12′″. The size of the images 20′″ remains the same but the position or location of the image 20′″ on each absorbent article 12′″ is different. Each image 20′″ is registered in a different machine and cross direction relative to the image printed on each adjacent absorbent article 12′″. The ability to print variable and diverse images 20′″ on each adjacent absorbent article 12′″ and to vary the frequency of a repeatable pattern is now possible using digital technology.

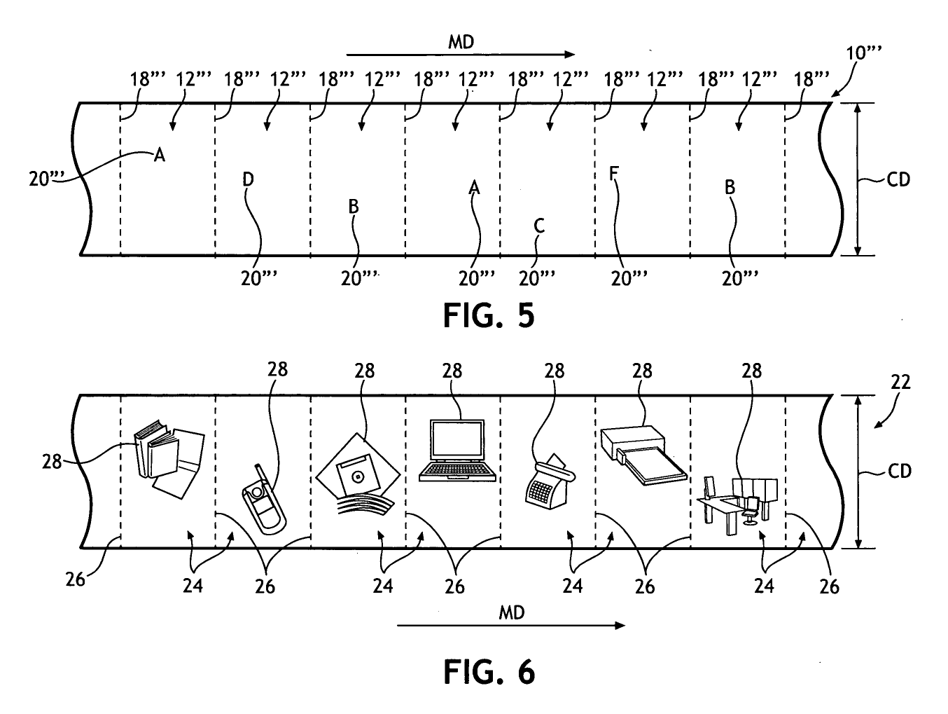

Turning now to FIG. 6, still another embodiment is shown of a strip of absorbent material 22, such as a strip of bath tissue, capable of being divided into a plurality of absorbent articles 24 along lines of perforations 26. Each of the absorbent articles 24 contains an image 28. The images 28 create a repeating pattern of distinct images 28 every n absorbent article 24, wherein n is desirably greater than 10. Since only seven complete absorbent articles 24 are shown, one is not able to view the repeating pattern. It should be noted that the value n can be set at a higher number than the total number of absorbent articles 24 present on the strip of absorbent material 22 such that no repeating pattern is visible to the ultimate consumer, if desired. In FIG. 6, one will readily notice that a single image 28, in the form of a graphical symbol, is printed on each of the absorbent articles 24. The size of the images 28 varies as well as the position or location of the image 28 on each of the absorbent articles 24. Each image 28 is registered in a different machine and cross direction relative to each adjacent absorbent article 24. The ability to print variable and diverse images 28 on each adjacent absorbent article 24 and to vary the frequency of a repeatable pattern is now possible using digital technology.

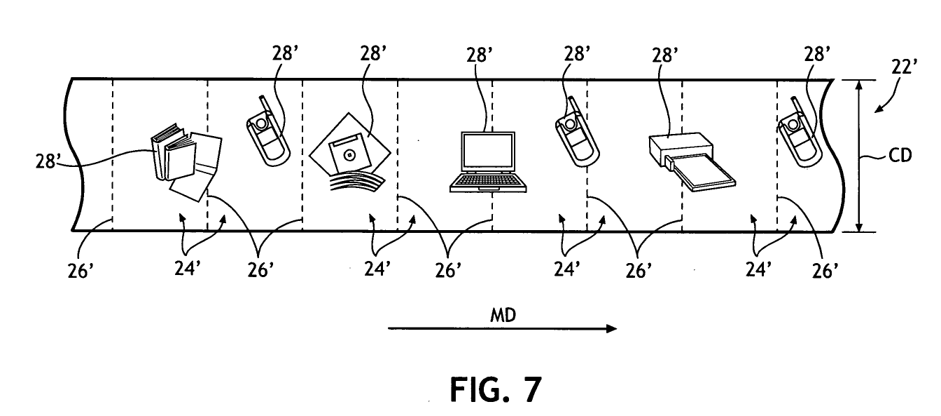

Turning now to FIG. 7, still another embodiment is shown of a strip of absorbent material 22′ capable of being divided into a plurality of absorbent articles 24′ along lines of perforations 26′. Each of the absorbent articles 24′ has at least a portion of an image 28′ printed thereon using digital technology. The strip of absorbent material 22′ can be a roll of bath tissue, paper towels, wipes, etc. The images 28′ desirably do not create a repeating pattern within the length of the strip of absorbent material 22′. In FIG. 7, one will readily notice that at least a portion of a single image 28′, in the form of a graphical symbol, is printed on each of the absorbent articles 24′. The size of the images 28′ varies as well as the position or location of the image 28′ on each of the absorbent articles 24′. Each image 28′ is registered in a different machine and cross direction relative to each adjacent absorbent article 24′. One will also notice that only seven images are present in the eight absorbent articles 24′. This means that each absorbent article 24′ does not contain a complete image 28′. The ability to print variable and diverse portions of images 28′ on each adjacent absorbent article 24′ is now possible using digital technology.

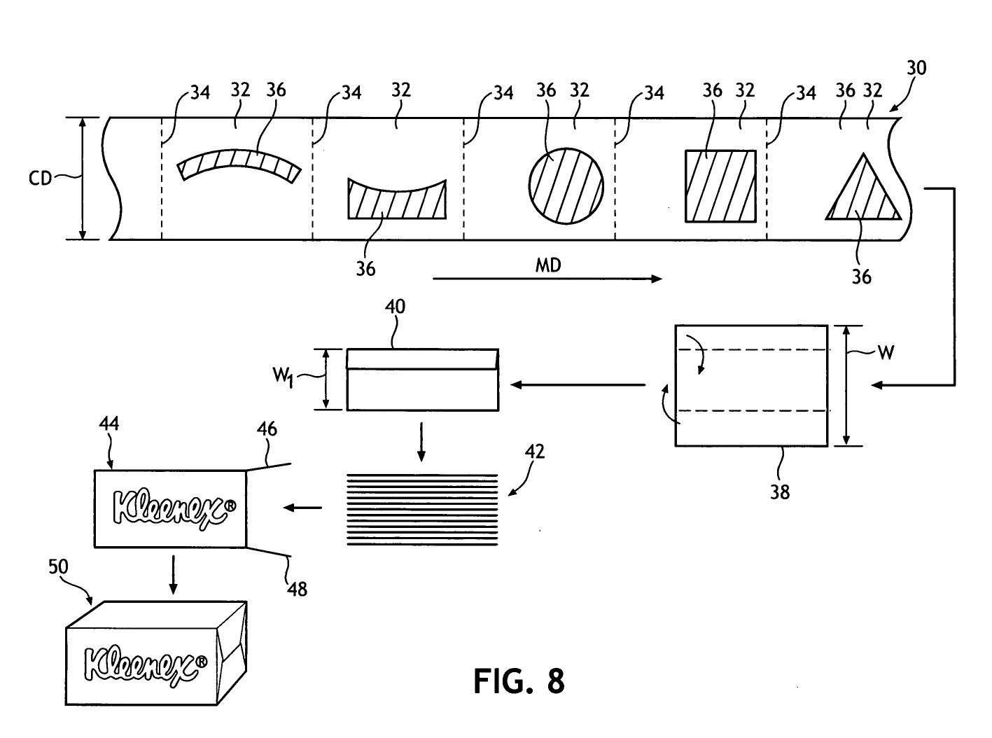

Turning now to FIG. 8, a strip of absorbent material 30, such as tissue, is shown which is capable of being divided into a plurality of absorbent articles 32. The strip of absorbent material 30 has a length extending in a machine direction MD and a width extending in a cross direction CD. The cross direction CD is aligned perpendicular to the machine direction MD. The strip of absorbent material 30 does not contain multiple lines of perforations but instead is designed to be severed or cut at spaced apart locations, designated by cut lines 34. Each of the absorbent articles 32 has a distinct image 36 printed thereon using digital technology. The distinct images 36 desireably do not create a repeating pattern within the length of the strip of absorbent material 30. After the strip, of absorbent material 30 is printed, the individual absorbent articles 38 are obtained by traversely cutting the strip 30 at the cut lines 34. Each of the individual absorbent articles 38 has a width w. The individual absorbent articles 38 are then folded to obtain a smaller absorbent article 40 having a reduced width dimension, designated w₁. The folded absorbent article 40 can represent a folded sheet of facial tissue. Multiple facial tissues 40 are then assembled into one or more stacks or piles 42. The stack(s) or pile(s) 42 is then inserted into a package 44. As an example, the package 44 can be a cardboard box having at least two end flaps 46 and 48 that can be opened to allow the stack 42 of facial tissues to be inserted into the package 44. The flaps 46 and 48 can then be closed and sealed, such as by an adhesive, to form a closed, filled package 50. The filled package 50 is now ready to be shipped to a retailer for sale to the ultimate consumer.

It should be noted that the package 44 can be formed from almost any kind of material known to those skilled in the art. Such materials include but are not limited to paper, cardboard, plastics, thermoplastics films such as polypropylene or polyethylene, laminates of paper and plastic, or any combination thereof. The package 44 can also be formed from other traditional materials such as wood, ferrous and non-ferrous metals, glass, etc.

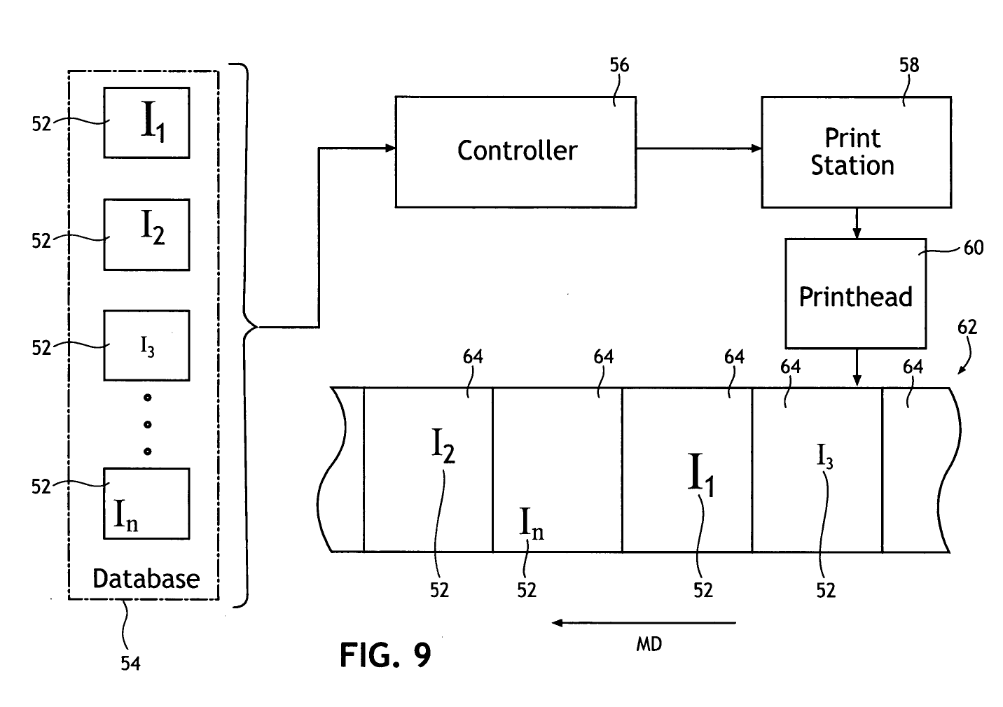

Referring now to FIG. 9, a flow diagram is shown which depicts one embodiment wherein a variety of distinct images 52 are stored in a database 54. The images 52 can be stored in any medium known to those skilled in the art. The images 52 can be any visual or perceived image in written, graphical, electronic, or art form. The images 52 can include but are not limited to letters, words, characters, text, symbols, icons, graphical representation of any object, device, animal or person, a painting, artwork, a photo, a printed image, a computer image, an animation, a Trademark, a Service mark, etc. The images can be formed from a single color or be formed from two, three, four or more different colors. Any desired color can be utilized. The image 52 can consist of a single color such as black, red, blue, green, yellow, etc., or it can include shades of a single color. The image 52 can also contain multiple colors and/or shading of one or more of the multiple colors.

The database 54 can be stored in a computer, on a disk or on an independent server that is electronically connected to one or more computers.

Alternatively, the database 54 can be located on the hard drive of a computer. One or more of the images 52 can be selected by a person who operates the computer. The information imputed into the computer will be electronically conveyed to a controller 56. The controller 56 can itself be a computer, microprocessor or some other type of programmable logic unit known to those skilled in the art. The controller 56 is, in turn, electronically connected to a print station 58. The print station 58 can also be a computer which selects an ink composition, the required colors, etc. The print station 58 controls a print head 60 such that the preselected images 52 can be created and printed onto the strip of absorbent material 62. The printed images 52 will be printed at a desired position or location, in a desired size, in a predetermined color(s) and at a registration such that any image 52 will be correctly present on any of the absorbent articles 64.

The print station 58 and the print head 60 are normally part of the ink jet printing system. An ink jet printer typically includes an ink jet print head that has a plurality of orifices (not shown). An ink composition made according to preselected criteria may be expelled from one or more of the orifices thus exiting the print head 60. Drops of the ink composition then travel a throw distance between the print head 60 and the moving strip of absorbent material 62. The orifices of the print head 60 may be aligned in a single row or may be formed having various patterns. The ink composition may be expelled from the orifices either simultaneously or through selected orifices at any given time. For many applications, the throw distance from the print head 60 to the surface of the strip of absorbent material 62 onto which the ink composition is applied is typically less than about 15 millimeters(mm), and commonly is less than about 5 mm.

According to the present invention, any suitable ink jet printing device can be used for applying ink compositions to the strip of absorbent material 62.

An ink jet printer is a non-impact printer that does not physically touch the strip of absorbent material 62. The ink composition to be applied to the strip of absorbent material 62 is in the form of discrete droplets. The size of the droplets can be varied as desired. Furthermore, placement of the droplets on the strip of absorbent material 62 can be precisely controlled using the controller 56. Because the ink jet printers do not contact the strip of absorbent material 62, the non-impact printers limit the spread of the ink composition once applied in the x, y and z directions and allow for the fine and discrete drops to remain intact on the strip of absorbent material 62. Drop size and the pattern used to apply the ink composition can be varied to allow for optimal sensory properties on the strip of absorbent material 62, such as softness, while still allowing for absorbency. Furthermore, non-impact printers can also be adjusted in order to control the amount of surface penetration that occurs when the ink composition contacts the strip of absorbent material 62. For example, in some applications, it is desired for the ink composition being applied to the strip of absorbent material 62 to remain on the surface and not migrate into the interior portions of the strip of absorbent material 62.

It should be noted that the ink composition can be altered to include additives that a manufacturer may wish to add to his absorbent articles 64. For instance, the additives can be a topical preparation that improves the physical properties of the strip of absorbent material 62. The additives can also be an anti- bacterial agent, a medicinal compound, an anti-acne agent, an anti-microbial agent, an anti-fungal agent, an antiseptic, an antioxidant, a softener, a cosmetic astringent, a drug astringent, an aiological agent, an emollient, an external analgesic, a humectant, a moisturizing agent, a skin conditioning agent, a skin exfoliating agent, a sunscreen, as well as mixtures thereof. These and other additives known to those skilled in the art can be utilized in varying amounts. For example, a bath tissue manufacturer may wish to add a softener, such as polysiloxane, to make his tissue feel softer to the touch.

Lastly, it should be noted that a plurality of absorbent articles 12 can be enclosed or contained in a package or some other type of product offering prior to being shipped to a retailer for sale. A product offering can be a closed or partially closed package or container. For example, an arrangement of absorbent articles 12 in a basket or on a tray will qualify as a product offering. The absorbent articles 12 can be formed into a roll, such as a roll of bath tissue or a roll of paper towels, or they can be folded, stacked, sorted or somehow arranged into one or more stacks, piles or bundles, such as facial tissue, wet wipes or individual towels, before they are packaged. It is also possible to position one or more different kinds of absorbent articles 12 into a single package or product offering, if desired.

### Method

The method of printing at least one distinct image on a first major surface of a strip of absorbent material is also disclosed. The method includes printing the at least one distinct image using digital printing technology. In addition, the strip of absorbent material is capable of being divided into a plurality of absorbent articles. This will allow the images to fully appear on each individual absorbent article or extend between one or more adjacent absorbent articles. For example, a single image could extend or cover two, three or more individual absorbent articles that are situated adjacent to one another.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A strip of absorbent material capable of being divided into a plurality of absorbent articles, each of said absorbent articles having a first major surface and at least one distinct image printed on said first major surface using digital printing technology.

2. The strip of absorbent material of claim 1 wherein said digital printing technology includes an ink jet printer connected to a computer having access to a database containing a variety of distinct images.

3. The strip of absorbent material of claim 1 wherein said distinct image printed on one of said absorbent articles is of a different size relative to said distinct image printed on an adjacent absorbent article.

4. The strip of absorbent material of claim 1 wherein said distinct image printed on one of said absorbent articles has a machine and cross direction registration which is different relative to said machine and cross direction registration of said distinct image printed on an adjacent absorbent article.

5. The strip of absorbent material of claim 1 wherein each of said absorbent articles is a folded sheet of facial tissue.

6. The strip of absorbent material of claim 1 wherein said absorbent articles are connected together by lines of perforations.

7. The strip of absorbent material of claim 6 wherein each of said connected absorbent articles is a bath tissue.

8. The strip of absorbent material of claim 6 wherein each of said absorbent articles is a paper towel.

9. The strip of absorbent material of claim 7 wherein said distinct image printed on one of said bath tissues is in registration with a distinct image printed on an adjacent bath tissue but is of a different size.

10. A strip of absorbent material capable of being divided into a plurality of absorbent articles, each of said absorbent articles having a first major surface, a second major surface, and at least one distinct image printed thereon using digital printing technology, said digital printing technology including an ink jet printer connected to a computer having access to a database containing a variety of distinct images, said distinct images being clearly visible on said first major surface, and said distinct image printed on one of said absorbent articles having a machine and cross direction registration which is different from a machine and cross direction registration of said distinct image printed on an adjacent absorbent article.

11. The strip of absorbent material of claim 10 wherein each of said distinct images is clearly visible on each of said first and second major surfaces.

12. The strip of absorbent material of claim 10 wherein each of said absorbent articles is a sheet of bath tissue connected together by a line of perforations and each of said distinct images is printed across one of said lines of perforations.

13. The strip of absorbent material of claim 10 wherein each of said absorbent articles is a sheet of bath tissue connected together by a line of perforations and each of said distinct images is spaced apart from said lines of perforations.

14. The strip of absorbent material of claim 10 wherein each of said absorbent articles has more than one image printed thereon.

15. The strip of absorbent material of claim 10 wherein each of said absorbent articles has only a partial image printed thereon.

16. A strip of absorbent material capable of being divided into a plurality of absorbent articles, said absorbent articles being contained in a package, each of said absorbent articles having a first major surface and at least one distinct image printed on said first major surface using digital printing technology, and said distinct image printed on one of said absorbent articles being of a different size relative to said distinct image printed on an adjacent absorbent article

17. The strip of absorbent material of claim 16 wherein each of said distinct images contains at least one color.

18. The strip of absorbent material of claim 17 wherein each of said distinct images contains at least two different colors.

19. The strip of absorbent material of claim 16 wherein each of said absorbent articles is a folded tissue sheet.

20. The strip of absorbent material of claim 16 wherein each of said absorbent articles is a bath tissue which is connected together by a line of perforations.

21. A method of printing at least one distinct image on a first major surface of a strip of absorbent material that is capable of being divided into a plurality of absorbent articles, said image being printed using digital printing technology.
